(19) United States
(12) Patent Application Publication
(10) Pub. No.: US 2009/0217403 A1
(43) Pub. Date: Aug. 27, 2009

(54) Means and methods for generating a t cell against an antigen of interest

(76) Inventor: Hergen Spits, San Francisco, CA (US)

Correspondence Address: TRASKBRITT, P.C., P.O. BOX 2550, SALT LAKE CITY, UT, 84110, US

(21) Appl. No.: 11/921,925

(22) PCT Filed: Jun. 6, 2006
(86) PCT No.: PCT/NL2006/000277

§ 371 Date: May 5, 2009

(30) Foreign Application Priority Data
Jun. 6, 2005 (EP) 05076308.5

(51) Int. Cl. A01K 67/027; C12N 5/10; C12N 5/08; C40B 40/02; C40B 30/04; A61K 48/00

(52) U.S. Cl. 800/18; 800/14; 435/455; 435/366; 506/14; 506/9; 424/93.21

(57) ABSTRACT

The invention provides a method for generating a T cell comprising a T cell receptor capable of specifically binding an antigen of interest, comprising: —providing a hematopoietic stem cell and/or a precursor cell of a T cell with a nucleic acid sequence comprising at least part of a rearranged gene encoding a TCR chain, or a functional equivalent thereof; and—allowing for differentiation of said stem cell and/or precursor cell and generation of at least one T cell derived from said stem cell and/or precursor cell.

The invention relates to the field of cell biology, in particular immunology.

An immunogenic substance is generally capable of eliciting two kinds of immune responses in a mammal: a humoral immune response leading to the production of antibodies and a cellular immune response predominantly enhancing the formation of reactive immune cells such as T cells. The cellular immune response is most effective in destroying infected cells and cancer cells. Cytotoxic T cells (CTLs, also called T killer cells) are capable of destroying cells displaying an epitope bound to a major histocompatibility complex (MHC) protein. An epitope presented to a cytotoxic T cell is recognized by said T cell through its T cell receptor (TCR): a transmembrane protein comprising two chains which are joined to each other, most often by a disulfide bond. The T cell receptors of most T cells comprise a TCR alpha chain linked to a TCR beta chain, whereas some T cells express receptors comprising a TCR gamma chain linked to a TCR delta chain. A T cell receptor is encoded by multiple V, D and J gene segments. During early T cell differentiation, a TCR alpha or gamma chain is formed by recombination of a V and a J gene segment while a TCR beta or delta chain is formed by recombination of a V, D and J gene segment. Shuffling of numerous V, J and D segments results in a high diversity of T cell receptors.

T cells are generated in the thymus. Haematopoietic precursor cells enter the thymus, followed by a cascade of reactions resulting in the formation of T cells with rearranged T cell receptor genes. Subsequently, selection of T cells takes place in the thymus: T cells capable of selectively binding foreign antigens are released while T cells which are specific for self antigens (also called autoantigens) are in principle destroyed in order to avoid autoimmune reactions. This system, referred to as negative selection, is not airtight and additional mechanisms are present in order to suppress immune reactions against autoantigens in the periphery through suppression mediated by so called T regulatory cells.

As a result of negative selection in the thymus and the action of T regulatory cells, many kinds of tumors are not or barely attacked by a cellular immune response. Antigens displayed on tumor cells are often identical to, or slightly different from, self antigens. This is especially the case for non virus-induced tumors. Because of the negative selection in the thymus against T cells having self antigen-specificity and the suppression of auto-reactive T cells by T regulatory cells, T cells capable of binding self antigens displayed on a tumor with high affinity are not released or not activated. Only T cells with low affinity are occasionally capable of leaving the thymus, but such T cells are usually not capable of efficiently counteracting such tumor.

There are various other occasions during which T cell responses are not sufficient in combating a disorder. Prolonged exposure to a (small) amount of antigen can for instance result in tolerance for said antigen. Moreover, some pathogens such as viruses are capable of suppressing a host's immune system.

When a subject's immune response is not capable of effectively counteracting a disease, it is desired to provide said subject with additional protection. Amongst other things, T cells with a desired specificity and affinity would be suitable for combating a disorder. In order to obtain such T cells, a plurality of T cells is preferably provided and screened for a T cell capable of specifically binding an antigen of interest. In most cases a selected T cell is used as such or nucleic acid of its T cell receptor is used.

In the art a method for generating T cells de novo is described. WO 2004/0171148 A1 describes an in vitro system comprising a Notch ligand that induces T cell lineage commitment and differentiation, and additionally induces stage-specific progenitor expansion, TCR gene rearrangement and T cell differentiation by hematopoietic progenitors and embryonic stem cells. However, the frequency of T cells that bind to a given antigen, for example to a HLA tetramer complexed with an antigenic peptide, is very low making it difficult to isolate a T cell with a specificity for a given antigen of interest. As an alternative of generating T cells de novo, methods have been described that generate random libraries of T cell receptors. For instance, phage display libraries comprising phages displaying single-chain TCRs have been described (Willemsen, 2001). These libraries are non-specific, so that a large amount of T cell receptors have to be generated in order to be capable of screening such library for the presence of a TCR with a desired specificity and affinity with a reasonable chance of success.

Furthermore, WO 01/55366 describes a method for generating a T cell library wherein TCR genes are mutated using PCR assembly. A TCRbeta DNA is generated that contains a 30% mutational rate in its 7 amino acids CDR3 region.

Kranz and collaborators disclose the production of a library wherein the CDR3 region of Va TCR sequences are mutated and expressed in yeast (Holler et al, 2000; Holler et al, 2003). The library was expressed with the complementary beta chain and TCR with a higher affinity could be isolated.

A disadvantage of these known methods is that they are laborious and that TCRs are obtained that are no longer exclusively specific for the original antigen, but exhibit significant cross-reactivity against other antigens.

It is an object of the present invention to provide an alternative method for generating a T cell capable of specifically binding an antigen of interest. Preferably a plurality of T cells capable of specifically binding an antigen of interest is provided.

The invention provides a method for generating a T cell comprising a T cell receptor capable of specifically binding an antigen of interest, comprising:

providing a hematopoietic stem cell and/or a precursor cell of a T cell with a nucleic acid sequence comprising at least part of a rearranged gene encoding a TCR chain, or a functional equivalent thereof; and

allowing for differentiation of said stem cell and/or precursor cell and generation of at least one T cell derived from said stem cell and/or precursor cell.

According to the present invention, a hematopoietic stem cell or precursor cell is provided with a nucleic acid sequence comprising at least part of a rearranged gene of a TCR, preferably at least part of a beta chain or alpha chain gene, or a functional equivalent thereof. Subsequently, said stem cell and/or precursor cell is allowed to differentiate and T cells are allowed to develop. A plurality of T cells derived from said stem cell or precursor cell have at least part of one chain in common, encoded by (at least part of) the rearranged gene or functional equivalent with which said stem cell and/or precursor cell had been provided. The complementary chain has been formed naturally. Hence, a method of the invention allows for the production of a plurality of T cells which is diverse and which comprises a higher content of T cells capable of specifically binding a given antigen of interest as compared to random libraries in the art. Therefore, a smaller amount of T cells of the invention need to be screened for a desired characteristic, and the chance of success is increased, as compared to random libraries of the art. Moreover, a method of the invention is less laborious as compared to artificial mutating techniques of the art such as PCR assembly, and cross-reactivity against other kinds of antigens is at least in part avoided.

Dedicated libraries of the art make use of T cells having rearranged TCR alpha and beta chain genes. Subsequently, at least one of said genes is mutated by a conventional mutation technique. This results in T cells which all have the same alpha and beta chain, or the same gamma and delta chain, albeit these chains are somewhat mutated. A method of the invention however provides T cells with different combinations of TCR chains. For instance, if a hematopoietic stem cell or progenitor cell is provided with a rearranged beta chain, T cells are generated with different alpha chains because the natural shuffling of alpha chain V and J segments occurs. In this embodiment T cells with the same beta chain and different alpha V and J segments are generated. Hence, a method of the invention enables the production of a diverse T cell library with a relatively high content of T cells capable of specifically binding a certain antigen of interest.

A T cell is defined herein as any kind of T cell comprising a T cell receptor. Said T cell comprises any of the known subsets of T cell, preferably a cytotoxic T cell. A T cell receptor is capable of specifically binding an antigen of interest when said T cell receptor has a significant higher affinity for said antigen of interest then for other compounds, although said T cell receptor may occasionally be capable of non-specifically binding another compound with low affinity. A hematopoietic stem cell preferably comprises a cell capable of differentiating to cells of any hematopoietic lineage. A precursor cell of a T cell comprises a cell which is capable of differentiating into a T cell. A precursor cell is also called a progenitor cell. A precursor of a T cell is also called herein a T cell precursor. Said stem cell and/or precursor cell is preferably capable of differentiating into any kind of T cell, although this is not necessary as long as said stem cell and/or precursor cell is capable of differentiating into at least one kind of T cell. Said precursor cell preferably comprises a CD34+ precursor cell. CD34 is expressed on stem cells and lineage precursor cells such as T cell precursors.

Said stem cell and/or precursor cell preferably comprises at least one unarranged TCR chain gene. More preferably at least four TCR chain genes of said stem cell and/or said precursor cell are unarranged. Two complementary TCR chain genes are two genes encoding two TCR chains which are capable of being naturally joined to each other and forming a functional T cell receptor. For instance, a TCR alpha and beta chain are two complementary TCR chains. Likewise, a TCR gamma and delta chain are two complementary chains. A TCR chain gene is unarranged if said gene comprises at least two V segments and at least two J segments (and at least two D segments, if present). Such unarranged chain gene is preferably in the germ line configuration, meaning that all V, J (and D, if present) gene segments are in the same position as in the genome of (non-hematopoietic) cells wherein TCR genes are not, and will not be, rearranged.

A rearranged gene encoding a TCR chain comprises a TCR chain gene with at least two recombined gene segments. This means that at least two different kinds of TCR gene segments are joined to each other. A rearranged TCR alpha or gamma chain gene preferably comprises a recombined VJ region. This means that one specific V segment is joined to one specific J segment. Other V and J segments, as well as other nucleic acid naturally present between said specific V segment and said specific J segment in an unarranged gene, which nucleic acid is naturally spliced out during gene rearrangement, are preferably absent. In one embodiment said rearranged gene encoding a TCR chain comprises a TCR chain gene which is partly rearranged. This means that at least two gene segments are already recombined but at least one further rearrangement event is required in order to obtain a gene encoding a functional TCR chain. Preferably however said rearranged TCR alpha or gamma chain gene encodes a functional TCR alpha or gamma chain. A rearranged TCR beta or delta chain gene for instance comprises a recombined DJ region. This means that one specific D segment is joined to one specific J segment. Other D and J segments, as well as other nucleic acid naturally present between said specific D segment and said specific J segment in an unarranged gene, which nucleic acid is naturally spliced out during gene rearrangement, are preferably absent. Preferably, said rearranged TCR beta or delta chain gene comprises a recombined VDJ region, meaning that one specific V segment, one specific D segment and one specific J segment are operably linked. A rearranged TCR gene encoding a functional TCR chain is preferred and is also called a productively rearranged TCR gene.

A part of a rearranged gene encoding a TCR chain is defined as a part of said gene comprising at least one TCR chain gene segment such as for instance a V, D or J segment. Said part preferably comprises two different kinds of gene segments (such as a V and J gene segment, or a D and J gene segment) which are linked to each other such that a recombined region has been formed. Nucleic acid naturally present between said two regions in the germ line configuration, which nucleic acid is naturally spliced out during gene rearrangement, is preferably not present in said part. Said part preferably comprises a recombined VJ region and/or DJ region. Other gene segments of the same kind are preferably absent. For instance, if said part comprises a recombined VJ region, other V and J segments are preferably absent. Likewise, if said part comprises a recombined DJ region, other D and J segments are preferably absent. In one embodiment said part comprises a recombined VDJ region. In this embodiment other V, D and J segments are preferably absent.

A part of a rearranged gene encoding a TCR chain preferably encodes a functional part of a T cell receptor chain. A functional part of a TCR chain comprises at least 100 amino acid residues, preferably at least 200 amino acid residues, most preferably at least 300 amino acid residues.

Said functional part comprises at least one same property as said TCR chain in kind, not necessarily in amount. Said functional part preferably has the same binding specificity as said TCR chain, which means that said part is capable of joining at least part of a complementary TCR chain, where after the resulting molecule is capable of binding the same antigen as said TCR, although the binding affinity may be different. Said functional part preferably comprises a V region with a hypervariable domain. The length of said functional part is preferably at least 70%, more preferably at least 80%, even more preferably at least 90%, most preferably at least 95% of the length of said TCR chain. A functional part with a longer length allows for the production of a functional part even more closely resembling said TCR chain. A functional part of a TCR chain preferably comprises at most one, or two, preferably at most 10, amino acid residues less than said TCR chain. A complex comprising said functional part linked to at least part of a complementary TCR chain preferably has essentially the same three-dimensional structure as said TCR.

A functional equivalent of a nucleic acid sequence of the invention is defined as a molecule encoding at least a functional part of a TCR chain and/or encoding a functional equivalent of at least part of a TCR chain. A functional equivalent of a nucleic acid sequence of the invention for instance comprises a nucleic acid analogue. Nucleic acid analogues are well known in the art.

In one embodiment said functional equivalent of a nucleic acid sequence of the invention comprises a nucleic acid sequence encoding at least a functional part of a TCR chain. In another embodiment said functional equivalent of a nucleic acid sequence of the invention comprises a nucleic acid sequence encoding a functional equivalent of at least part of a TCR chain. A functional equivalent of at least part of a TCR chain is a molecule comprising at least one same property as said at least part of a TCR chain in kind, not necessarily in amount. Said functional equivalent of at least part of a TCR chain is preferably capable of joining at least part of a complementary TCR chain, where after the resulting molecule is capable of binding the same antigen as said TCR, although the binding affinity may be different. In the art many methods are available for designing and generating a functional equivalent of a TCR chain. For instance, said functional equivalent is designed and/or generated by modification of a TCR chain sequence. In one embodiment conservative amino acid substitution is applied.

The art provides various ways for introducing a nucleic acid sequence into a stem cell or progenitor cell. In one embodiment said nucleic acid sequence is introduced into the cell by a vector, preferably a retroviral vector. In one embodiment a lentiviral vector is used because lentiviral vectors are capable of efficiently transducing dividing and non-dividing cells. Hematopoietic stem cells are well transduced by lentiviral vectors.

A method of the invention enables production of a T cell comprising a T cell receptor capable of specifically binding a gene of interest. Once said T cell has been generated, it is preferably at least in part isolated for further use. A method of the invention therefore preferably further comprises at least in part isolating at least one T cell derived from said stem cell and/or precursor cell, which T cell is capable of specifically binding an antigen of interest. Methods for at least in part isolating a T cell are known in the art. A sample is for instance enriched in T cells by flow cytometry cell sorting. Furthermore, a T cell capable of specifically binding a specific antigen of interest is preferably at least in part isolated using a binding assay with said antigen of interest, and/or with a functional part, derivative and/or analogue of said antigen. A functional part of an antigen is defined as a part of said antigen which is capable of being specifically recognized by a TCR when presented in a specific MHC context. Said functional part is preferably about 8-12 amino acids, more preferably about 9-11 amino acids long. A derivative and/or analogue of an antigen are defined as a modified sequence of at least part of said antigen, which modified sequence is still capable of being recognized by a TCR which is specific for said antigen. The art provides many alternative methods for isolating T cells.

In order to generate a T cell capable of specifically binding an antigen of interest, a hematopoietic stem cell or a precursor cell of a T cell is preferably provided with at least part of a rearranged TCR chain gene encoding at least part of a TCR chain with a specificity for a given antigen. In that case it is possible to generate a plurality of T cells, also called a library, comprising one chain derived from said rearranged TCR chain gene and a plurality of different complementary chains. For instance, if said stem cell and/or precursor cell is provided with at least part of a rearranged TCR beta chain gene, a library of T cells comprising said at least part of said beta chain and a variety of alpha chains is obtained. Since said beta chain is biased to specifically binding said antigen of interest, said library will comprise a (relatively) high content of T cells capable of specifically binding said antigen of interest. Generated T cells are preferably subsequently screened for their ability to bind said antigen of interest and at least one T cell with a desired characteristic is preferably selected. The invention therefore provides a method of the invention, wherein said rearranged gene encoding at least a functional part of a TCR chain is derived from a first T cell receptor, said first T cell receptor being capable of binding said antigen of interest.

Various methods are available in the art for obtaining a rearranged TCR chain gene from a T cell. Preferably, T cell nucleic acid is isolated and a rearranged gene of interest is obtained by a nucleic acid amplification reaction such as PCR with at least one specific primer and/or probe. Of course, once a sequence of a rearranged gene of interest is known, it is possible to artificially generate said rearranged gene, using any method known in the art.

A T cell comprising a T cell receptor with a desired property is preferably selected. For instance, a T cell which is capable of recognizing said antigen of interest when presented in a specific MHC context is selected. Additionally, or alternatively, it is possible to select a T cell with a desired function. For instance, a CTL and/or a T helper cell is selected.

In a preferred embodiment a T cell is selected which comprises a T cell receptor which is capable of binding said antigen of interest with a higher affinity as compared to said first T cell receptor from which said rearranged gene is derived. This embodiment is particularly preferred when a T cell capable of specifically binding a self antigen is desired, and/or when a T cell is desired which is capable of specifically binding a non-self antigen for which an immune system has become tolerant. One important application is treatment of a tumor. Circulating T cells capable of binding a tumor-associated self antigen with a high affinity are usually not found. However, T cells capable of binding a tumor-associated self antigen with a (very) low affinity are regularly found.

According to one embodiment, a low affinity T cell with a desired specificity (preferably against a tumor associated antigen) is isolated. Subsequently, at least one rearranged TCR chain gene from said low affinity T cell is isolated and/or identified. A nucleic acid sequence encoding one rearranged TCR chain from said low affinity T cell is provided, and introduced into a hematopoietic stem cell and/or a precursor cell of a T cell. Subsequently, said stem cell and/or precursor cell is allowed to differentiate and T cells derived from said stem cell and/or precursor cell are isolated. Finally, said T cells are incubated with an antigen for which said original low affinity T cell was specific. Alternatively, or additionally, said T cells of the invention are incubated with an immunogenic part, derivative and/or analogue of said antigen. It is preferably determined whether a T cell has been generated with a higher affinity for said antigen, as compared to said original low affinity T cell. This way, it has become possible to generate a T cell with a specificity for the same kind of antigen, but with a higher affinity, as compared to an originally available T cell.

A method of the invention is preferably performed with a human stem cell and/or a human precursor cell, in order to generate human T cells. A human T cell with a desired property, or a nucleic acid derived from said human T cell, is preferably used in order to provide a human individual with the property of attacking a cell expressing an unwanted antigen such as a tumor antigen, or in order to enhance a human individual's immune response against said cell expressing an unwanted antigen.

A hematopoietic stem cell and/or a precursor cell is preferably provided with a rearranged TCR chain gene that is derived from the same species, so that a T cell is obtained without foreign nucleic acid sequences derived from another species. A possible immune response against said T cell in an individual of the same species as said hematopoietic cell and/or precursor cell, is at least in part avoided.

Most preferably, said hematopoietic stem cell and/or precursor cell and said rearranged gene encoding at least a functional part of a TCR chain are human. This embodiment is particularly suitable for therapeutic applications in human beings.

In a preferred embodiment a hematopoietic stem cell and/or a precursor cell of a T cell is provided with a nucleic acid sequence comprising at least part of a rearranged alpha or beta chain gene of a T cell receptor. The majority of T cells found in mammals such as humans comprise T cell receptors with a TCR alpha and beta chain. Hence, many T cells derived from a stem cell or precursor cell which has been provided with a rearranged TCR alpha or beta chain gene will contain said rearranged gene. In a most preferred embodiment a hematopoietic stem cell and/or a precursor cell of a T cell is provided with a nucleic acid sequence comprising at least part of a rearranged TCR beta chain gene. A rearranged beta chain gene is preferred because in nature a beta chain gene is rearranged before rearrangement of an alpha chain gene. Hence, if said stem cell or precursor cell is provided with a rearranged beta chain gene, the natural situation is more closely imitated. This is however not necessary: in another embodiment said stem cell and/or precursor cell is provided with a nucleic acid sequence comprising a rearranged alpha chain of a T cell receptor.

As explained above, a method of the invention is preferably used for generating a plurality of T cells, also called a library, which is subsequently preferably screened for at least one desired characteristic, such as binding specificity, binding affinity and/or stability. The invention thus provides a method of the invention wherein a plurality of T cells derived from said stem cell and/or precursor cell is obtained. Preferably a plurality of T cells with different antigen binding affinities is obtained.

Once a hematopoietic stem cell and/or a precursor cell of a T cell has been provided with a nucleic acid sequence comprising at least part of a rearranged gene encoding a TCR chain, said stem cell or precursor cell is allowed to multiply and/or differentiate. This is possible in a variety of circumstances. In one embodiment, said stem cell and/or precursor cell multiplies and/or differentiates in vitro. Hence, in this embodiment a T cell is generated in vitro. This is for instance done using an in vitro system as described in WO 2004/0171148 A1, said system comprising a Notch ligand that induces T cell lineage commitment and differentiation of hematopoietic progenitor cells and embryonic stem cells. In another embodiment, a fetal thymus organ culture (FTOC) system as described in (Res et al, Blood. 87: 5196-5206 1996) is used. Alternative methods known in the art for multiplying and differentiating hematopoietic stem cells and/or progenitor cells are suitable as well.

In another embodiment, said stem cell and/or precursor cell is allowed to multiply and/or differentiate in vivo. Hence, in this embodiment a T cell is generated in vivo, using a non-human animal. This allows for generation of T cells of interest which are often not of therapeutic value for said non-human animal, but which are harvested and for instance used for therapeutic applications in humans. T cells produced in said non-human animal are preferably isolated and screened for a desired property. A selected T cell, and/or its nucleic acid, is subsequently isolated for further use. It is possible to provide an animal's endogenous hematopoietic stem cell and/or precursor cell of a T cell with a nucleic acid sequence comprising at least part of a rearranged TCR chain. It is however preferred to provide a non-human animal such as a rodent, preferably a rat or a mouse with a human hematopoietic stem cell and/or a human precursor cell of a T cell, in order to generate human T cells. In one embodiment a stem cell and/or precursor cell is provided with a nucleic acid sequence comprising at least part of a rearranged gene before said cell is provided to said non-human animal. Said stem cell and/or precursor cell is for instance provided with said nucleic acid sequence by a (retro)viral vector, preferably a lentiviral vector, after which said stem cell and/or precursor cell is provided to said non-human animal. In another embodiment, said stem cell and/or precursor cell is provided with said nucleic acid sequence comprising said rearranged gene after it has been provided to said non-human animal. Preferably a lentiviral vector is used.

In order to enhance the formation of T cells capable of specifically binding an antigen of interest, said non-human animal is preferably provided with said antigen of interest after said at least one T cell is generated. Said antigen of interest is preferably not endogenously present within said non-human animal. The presence of a (foreign) antigen of interest in an animal triggers the formation T cells specific for said antigen. In a preferred embodiment a non-human animal is provided with a human antigen. More preferably, said antigen comprises at least an immunogenic part of a non-hematopoietic human autoantigen, a non-hematopoietic tumor-associated antigen and/or a non-hematopoietic antigen expressed on malignant cells. In the thymus of a non-human animal which has been provided with a human stem cell and/or human precursor cell of the invention, human T cells and human dendritic cells are present. T cells with high-affinity TCRs specific for self-antigens that are present on hematopoietic cells will therefore be deleted. However, in view of the absence of non-hematopoietic human cells of said non-human animal, T cells with high-affinity TCRs specific for self-antigens that are absent from hematopoietic cells will not be deleted. Since the thymus of a non-human animal does not select against T cells with a high affinity for human (auto)antigens that are not expressed on hematopoietic cells, such T cells are capable of leaving the thymus and circulating within said non-human animal. Hence, a method of the invention allows for production of a T cell capable of specifically binding a non-hematopoietic human (auto)antigen with high affinity. Preferably, a plurality of T cells specific for a non-hematopoietic human (auto)antigen is produced. Said T cells are preferably isolated and screened for the presence of a T cell with at least one desired characteristic.

As described above, T cells in general are for instance isolated by flow cytometry cell sorting. However, a T cell capable of specifically binding an antigen of interest is preferably obtained with a binding assay using said antigen of interest or a functional part, derivative and/or analogue thereof. Said antigen or functional part, derivative or analogue thereof is preferably presented to a T cell in the right MHC context. In one embodiment, T cells obtained by a method of the invention are incubated with MHC-peptide complexes. Unbound T cells are subsequently washed away and bound T cells are isolated, for instance by cell sorting using magnetic beads and/or flow cytometry. In one embodiment multimeric MHC complexes, such as MHC tetramers and/or MHC-Ig dimers are used, for instance as described in (Altman et al, 1996) and (Schneck, 2000). Assay systems that use T cell activation as a readout system are also suitable. Such assay systems are well known in the art and do not need further explanation here.

When non-endogenous T cells such as for instance human T cells are produced in a non-human animal, said animal's immune system is preferably at least partly impaired, in order to at least partly avoid animal immune responses against said non-endogenous T cells. This is for instance accomplished by irradiating said animal before it is provided with a non endogenous (preferably human) stem cell and/or precursor cell. Preferably, said non-human animal is essentially devoid of at least one kind of endogenous hematopoietic cell. Said non-human animal is preferably devoid of endogenous B cells, endogenous T cells and/or endogenous natural killer (NK) cells. In one embodiment a knock out non-human animal is used which is devoid of at least one gene responsible for said animal's immune response. A knock out non-human animal devoid of at least one gene involved in the production of endogenous B cells, endogenous T cells and/or endogenous natural killer cells is preferred. A knock out animal is for instance produced by gene silencing or by introducing mutations using methods well known in the art. Gene silencing is for instance performed by providing said animal with a compound capable of specifically binding (an expression product of) said gene. Said compound for instance comprises a protein or antisense RNA. Alternatively, or additionally, gene silencing is performed using small interfering RNAs. Small interfering RNAs (siRNAs) of approximately 21-23 base pairs (bp) are preferably cleaved from double-stranded precursor RNAs by the RnaseIII-like enzyme DICER. These siRNAs are capable of associating with various proteins to form the RNA-induced silencing complex (RISC), harbouring nuclease and helicase activity. The antisense strand of the siRNA guides the RISC to the complementary target RNA, and the nuclease component cleaves the target RNA in a sequence-specific manner. Hence, double stranded RNA is capable of inducing degradation of the homologous single stranded RNA in a cell. Synthetic siRNAs of about 21 bp are shown to efficiently induce RNAi-mediated gene silencing when introduced into a cell. In one embodiment, RNAi are induced in mammalian cells by intracellularly expressed short hairpin RNAs (shRNAs), preferably with a length of 19 bp, with a small loop.

It is also possible to induce at least one mutation in at least one gene involved in the production of hematopoietic cells. Mutations are for instance induced using site specific mutagenesis. Many alternative methods for producing a knock out non-human animal are known in the art which do not need further explanation here. Hence, a method of the invention is provided wherein said non-human animal is essentially devoid of at least one kind of endogenous hematopoietic cell.

In a preferred embodiment a non-human animal is used which is essentially devoid of endogenous B cells, endogenous T cells, and/or endogenous natural killer (NK) cells. Said non-human animal preferably comprises a mouse, more preferably a RAG2^{-/-} yc^{-/-} mouse, as described in (Kirberg et al, 1997, incorporated herein by reference) and (Weijer et al, 2002, incorporated herein by reference) which is a double mutant strain lacking B, T and NK cells. Transplantation of human stem cells and/or human hematopoietic precursor cells into said mouse results in a mouse with a human hematopoietic system (Weijer et al, 2002; Traggiai et al, 2004; Gimeno et al, 2004 (incorporated herein by reference)). Said mouse is very suitable for use in a method of the present invention, since generated human T cells are not, or to a little extent, attacked by murine immune responses.

In one aspect of the invention said stem cell and/or precursor cell is administered to said non-human animal within one week after birth. Preferably, said stem cell and/or precursor cell is administered to said non-human animal within three days after birth, more preferably within one day after birth. It has been demonstrated by the present inventors that early injection results in increased T cell engraftment.

The invention furthermore provides a T cell identified and/or obtainable by a method of the invention as well as at least part of a nucleic acid encoding a T cell receptor of said T cell. Said nucleic acid preferably comprises at least one rearranged TCR chain. Preferably, said T cell comprises a human T cell. As described above, a plurality of T cells is preferably generated. Said plurality of T cells, also called a library, is suitable for screening in order to select a T cell with a desired characteristic. A T cell capable of specifically binding an antigen of interest is for instance selected by incubating said library with said antigen of interest, a peptide derived from said antigen of interest, or with an immunogenic part, derivative or analogue thereof, in a suitable MHC context and selecting a T cell bound to said antigen, peptide, immunogenic part, derivative or analogue. A library comprising a plurality of T cells obtainable by a method of the invention is therefore also provided. Said library preferably comprises T cells having T cell receptors with different antigen binding affinities. The invention furthermore provides a method for selecting a T cell comprising a T cell receptor capable of specifically binding an antigen of interest, comprising contacting said antigen of interest or a functional part, derivative and/or analogue thereof with a library according to the invention and selecting a T cell bound to said antigen or functional part, derivative and/or analogue.

Once a T cell of the invention has been obtained, at least one nucleic acid sequence encoding at least part of said T cell's T cell receptor is preferably obtained. This is for instance performed by generation of TCR cDNA using TCR RNA as a template, using at least one specific primer (for instance by a reverse transcriptase reaction). Said nucleic acid preferably comprises at least one rearranged TCR chain. More preferably, at least two rearranged TCR chain genes are obtained. Most preferably two rearranged genes encoding two complementary TCR chains are obtained. Said at least one nucleic acid sequence is preferably brought into a suitable host cell in order to provide said host cell with the capability of specifically binding said antigen of interest. This is for instance performed by (retro)viral transduction of said host cell. Said host cell preferably comprises a T cell. In one preferred embodiment said host cell comprises a T cell derived from an individual. Said T cell is preferably subsequently (re)introduced into said individual in order to provide said individual with (additional) capability of at least in part attacking an undesired antigen of interest. Said T cell is preferably derived from said individual in order to avoid an immunogenic response against said T cell.

This embodiment is particularly suitable for counteracting a tumor-related disease, because tumor cells often comprise self antigens at their surface and are therefore often not attacked by an individual's own immune system. This embodiment is also suitable in case of undesired tolerance against a non self antigen.

In one embodiment said T cell is introduced into another individual. At least, said other individual should be matched for an HLA molecule that is used by said T cell.

In one preferred embodiment a low affinity T cell from an individual, preferably a human individual, with a specificity against an antigen of interest, preferably a self antigen, is obtained. A nucleic acid sequence encoding one rearranged TCR chain of said low affinity T cell is subsequently obtained. Said nucleic acid sequence is introduced into a hematopoietic stem cell or a T cell precursor cell of a subject of the same species (preferably a human hematopoietic stem cell or a human precursor cell of a T cell). Said stem cell or precursor cell is allowed to differentiate and T cells derived from said stem cell or precursor cell are obtained. In this embodiment, said T cells are subsequently incubated with said antigen of interest (preferably a human self antigen) and T cells capable of specifically binding said antigen are obtained. Subsequently, a T cell capable of binding said antigen with a higher affinity as compared to said original low affinity T cell is preferably isolated. According to this embodiment, nucleic acid comprising a rearranged gene encoding at least one TCR chain, preferably comprising two complementary rearranged genes encoding both TCR chains, is obtained and introduced into a T cell derived from an individual which is matched for an HLA molecule that is used by said T cell. Said T cell is reintroduced into said individual. This way, said individual is provided with an (enhanced) immune response against an undesired antigen, such as for instance a tumor-related (self) antigen. In one embodiment nucleic acid comprising a rearranged gene encoding at least one TCR chain, preferably comprising two complementary rearranged genes encoding both TCR chains, is obtained and introduced into a T cell derived from the same individual from whom said low affinity T cell was obtained.

The invention thus provides a method according of the invention, further comprising:

obtaining a nucleic acid sequence encoding said T cell receptor; and

providing a suitable host cell with said nucleic acid sequence.

An isolated host cell capable of specifically binding an antigen of interest obtainable by a method of the invention is of course also herewith provided. Said host cell preferably comprises a T cell, more preferably a human T cell in order to allow therapeutic applications for human individuals, as explained above. One important application comprises providing a T cell from an individual with the capability of binding an antigen of interest with a desired affinity. The invention therefore also provides a method for providing a T cell with the capability of binding an antigen of interest with a desired affinity, comprising providing said T cell with a nucleic acid sequence encoding a T cell receptor obtainable by a method according to the invention. A T cell capable of binding an antigen of interest with a desired affinity obtainable by a method of the invention is also provided. Said T cell is preferably administered to an individual in order to provide said individual with a capability of generating an immune response against an antigen of interest or to enhance said individual's capability of generating an immune response against an antigen of interest. A method for providing an individual with an (enhanced) capability of generating an immune response against an antigen of interest, comprising providing said individual with at least one T cell and/or host cell obtainable by a method of the invention, is therefore also provided. Preferably, said T cell and/or host cell is derived from said individual, in order to efficiently avoid an immunogenic response against said T cell and/or host cell. At least, said subject should be matched for an HLA molecule that is utilized by said T cell and/or host cell.

A host cell of the invention or a nucleic acid sequence encoding a T cell receptor obtainable by a method of the invention is thus suitable for therapy. The invention therefore furthermore provides a host cell of the invention or a nucleic acid sequence encoding a T cell receptor obtainable by a method of the invention for use as a medicament. Said therapy preferably comprises a tumor-related disease, because a cellular response against tumor cells is often low. One embodiment therefore provides a use of a host cell of the invention or a nucleic acid sequence encoding a T cell receptor obtainable by a method of the invention, for the preparation of a medicament against a tumor-related disease.

An isolated stem cell and/or precursor cell of a T cell, said stem cell and/or precursor cell being provided with a nucleic acid sequence comprising at least part of a rearranged gene encoding a TCR chain of a T cell receptor is also herewith provided. Said nucleic acid is preferably derived from a T cell receptor which is capable of specifically binding at least an immunogenic part of a human autoantigen, a tumor-associated antigen and/or an antigen expressed on malignant cells. In one embodiment said stem cell and/or precursor cell is allowed to differentiate in a non-human animal. T cells derived from said stem cell and/or precursor cell are subsequently present in said non-human animal. The invention therefore provides a non-human animal comprising a stem cell, precursor cell and/or T cell according to the invention. Said non-human animal preferably comprises a RAG2^{-/-} yc^{-/-} mouse. As explained before, a RAG2^{-/-} yc^{-/-} mouse is essentially devoid of murine T, B and NK cells and therefore does not, or to a little extent, elicit an immune response against (foreign) stem cells, precursor cells and/or T cells of the invention.

The invention is further illustrated by the following examples. The examples do not limit the scope of the invention in any way.

### EXAMPLES

### Material and Methods

Preparation of Haematopoietic Progenitor Cells from Fetal Liver

Early haematopoietic progenitor human cells were isolated from foetal liver obtained from elective abortions, with gestational age ranging from 14 to 20 weeks. The use of this human material was approved by the Medical Ethical Committees of the Academic Medical Centre of the University of Amsterdam (AMC-UvA) and was contingent on informed consent.

Single cell suspensions were prepared from fetal liver and mononuclear cells were isolated by density gradient centrifugation over Lymphoprep Ficoll-Hypaque (Nycomed Pharma). Enrichment of CD34⁺ progenitor cells (>98% pure) was performed by using the CD34 Progenitor Cell Isolation Kit (foetal liver), or the Undirect CD34 Progenitor Cell Isolation Kit (post-natal thymocytes), both from Miltenyi Biotech.

Isolation of CD34⁺ Cells from Postnatal Thymus.

The use of postnatal thymus tissue was approved by the Medical Ethical Committee of the Academic Medical Center. Thymocytes were obtained from surgical specimens removed from up to three years old children undergoing open-heart surgery. The tissue was disrupted by mechanical means and pressed through a stainless steel mesh to obtain a single cell suspension, which was left overnight at 4° C. The following day cells were isolated from a Ficoll-Hypaque (Lymphoprep; Nycomed Pharma, Oslo, Norway) density gradient. Subsequent CD34⁺ cells were enriched by immunomagnetic cell sorting, using a CD34 cell separation kit (varioMACS, Miltenyi Biotec). The CD34⁺ thymocytes were stained with anti-CD34 and anti-CD1a and separated into CD34⁺CD1a⁻ and CD34⁺CD1a⁺ populations by cell sorting using a FACSAria (BD).

### Constructs, Cell Lines, and Retroviral Production.

The OP9-control, OP9-DL1 cell lines were generated by transduction of the murine bone marrow stromal cell line OP9, (kindly provided by Dr. T. Nakano (Osaka University, Osaka, Japan) (Nakano et al., 1994) with respectively the empty LZRS IRES neo retroviral vector or with the LZRS IRES neo vector engineered to express DeltaLikel (DL1) (provided by Dr. L Parreira Instituto de Histologia e Embriologia, Faculdade de Medicina de Lisboa, Lisbon, Portugal). Transduced cells were selected on their resistance for neomycin by culturing for 2-3 weeks in the presence of 1.5 mg/ml Geneticin (G418, Invitrogen, CA) Cells were maintained in MEMα (Gibco Invitrogen) with 20% FCS. Retroviral supernatants were produced as described using the 293T based Phoenix packaging cell line (Kinsella et al., 1996).

Retroviral transduction and differentiation assays

For transduction experiments CD34⁺CD1a⁻ postnatal thymocytes cells were cultured overnight in Yssels medium supplemented with 5% NHS and 10 ng/ml SCF and 10 ng/ml IL-7. The following day the cells were incubated for 6 to 7 hours with virus supernatant in retronectin coated plates (30 μg/ml; TakaraBiomedicals, Otsu, Shiga, Japan). The following TCRs were used: aCMV TCR-AV19/BV21; aCMV TCR-AV18/BV13; aHA2.6 TCR-AV23/BV18 (provided by Dr. M. Heemskerk, LUMC, Leiden); aMART-1 TCR-AV25/BV12 provided by Dr. T. Schumacher, NKI, Amsterdam). All TCR chains were inserted in the retroviral pLZRS vector in tandem with GFP (TCR-AV genes) or YFP (TCR-BV genes) reporter genes. Amphotropic viruses were produced after FUGENE® (Roche) transfection of FGALV packaging cell line ((Kinsella et al., 1996), provided by Dr. Nolan). The virus-containing supernatants were passed through a 0.22 mm filter, aliquoted and kept at −80° C. development of T cells was assessed by co-culturing 50.000 CD34⁺ progenitor cells with 50.000 OP9 cells. Cultures were performed according to J-C. Zúcñiga-Pflücker (La Motte-Mohs et al., 2005) in MEMα medium (Gibco) with 20% FCS (Hyclone, Logan, ULT) supplemented with 5 ng/ml IL-7 and 5 ng/ml Flt3L. Medium and cytokines were refreshed every 2-3 days and progenitor cells were transferred to fresh stromal cells every 4-5 days of culture. Flow cytometric analyses were performed on an LSRII FACS analyzer (BD).

### Expansion T Cells and Cytotoxic Assays

T cells that developed in the OP9DL co-cultures were expanded using a feeder cell mixture consisting of irradiated allogeneic peripheral blood lymphocytes (106/ml), irradiated cells of the EBV transformed cell line JY (2×105/ml) and phytohemagglutinin (0.1 μg/ml) in Yssel's medium containing 2% human serum, exactly as described by Spits et al. 1982 and Yssel et al. 1984. Cytotoxic activity was determined by a standard 51Cr release assay using the EBV-transformed cell line ZIJL as target. To measure CMV-specific responses, ZIJL cells were transduced with a PP65-encoding DNA in the LZRS-IRES-GFP vector or were loaded with 10 μg/ml PP65-derived HLA-A2-binding peptide.

Generation of Humanized Rag2^{-/-}γc^{-/-} Mice with Enforced TCR Expression

H-2^{d} Rag2^{-/-}γc^{-/-} mice (Kirberg et al., 1997) were bred and maintained in isolators, and they were fed with autoclaved food and water. All manipulations of HIS-Rag/γc mice were performed under laminar flow. Mice with humanized immune system (HIS-Rag/γ_{c}) were generated as previously described (Gimeno et al., 2004). Briefly, newborn (<1 week old) Rag2^{-/-}γc^{-/-} mice received a sub-lethal (350rad) total body irradiation with a ¹³⁷Cs source, and were injected i.p. with 1-2.10⁶ TCR-transduced CD34⁺ human fetal liver cells. Cell suspensions were prepared in RPMI medium with 2% fetal calf serum, before flow cytometry analysis.

### Flow Cytometry Analysis

Cell suspensions were stained with anti-human monoclonal antibodies targeting the following cell surface markers: CD45, CD3, CD4, CD8, CD28, CD123/IL-3Ra, CD25/IL-2Rα, TCRαβ, TCRγδ, HLA-DR, CD5, CD7, from BD Bioscience, CD1a from Coulter-Immunotech, and BDCA2 from Miltenyi Biotech. All washings and reagent dilutions were done with PBS containing 2% fetal calf serum (FCS) and 0.02% sodium azide (NaN₃), and each step of staining was done at 4° C. in the dark for 20 minutes. Dead cells were excluded according to their light-scattering characteristics. All acquisitions were performed with a LSR-II (BD Bioscience) cytometer interfaced to FACS-Diva software system.

### Results

In Vitro Generation of CTL from Human Cd34+ Precursor Cells.

Zúnĩga-Pflücker and collaborators have demonstrated that human neonatal cord blood cells cultured with the murine stromal cell line OP9 that express the Notch ligand DL1 develop into T cells (La Motte-Mohs et al., 2005). We have confirmed these data and show that CD34+CD1a-thymic precursor cultured with OP9DL1 develop into mature T cells in 3-4 weeks (FIG. 1). The CD8+ T cells were functionally mature because they lacked CD1a. We have demonstrated previously that loss of CD1a in single positive thymocytes is accompanied by functional maturation (Res et al., 1996). Furthermore the CD8+ T cells could be expanded following culture with feeder cell mixture consisting of irradiated PBMC, irradiated JY cells and PHA. These cells mediate cytotoxic activities induced by anti-CD3 antibodies (results not shown). Although in the mouse it was reported that another Notch-1 ligand Jagged cannot support T cell development, we observed T cells development following coculture of CD34+CD1a-thymocyte precursors with OP9Jagged which was very similar to that induced by OP9DL1.

TCR Transfer into CD34+ Precursor Cells and Expression in the Mature T Cell Progeny

The experiment shown in FIG. 1 demonstrates that OP9DL1 and OP9Jagged cells can mediate differentiation of CD4+CD8+ cells into functional CD8+ T cells. Since generation of mature single positive CD8+ T cells requires an interaction between the TCR and its ligand we should assume that the mature CD8+ T cells were positively selected by interaction with MHC. To investigate whether this system can select T cells with a TCR with defined specificity we examined the capacity of OP9DL1 to support differentiation of stem cells that were transduced with HLA-A2-restricted TCRs (FIG. 2). In these experiments we used TCRs specific for the minor histocompatibility antigen HA-2 provided by Dr M. Heemskerk of the LUMC. Dr Heemskerk constructed the TCRα and β in separate vectors in the configuration TCRβ-IRES-ANGFR and TCRα-IRES-GFP. ΔNGFR is a signaling-incompetent mutant of the nerve growth factor receptor and its expression can be detected with a monoclonal anti-NGFR antibody. FIG. 3 demonstrates that OP9DL cells could support development of T cells expressing both the TCRα and β chain specific for the HA2/HLA-A2 complex. It is shown that the T cells expressing both the alpha and the beta chain of this TCR developed more rapid to mature CD8+CD1a-T cells than the control-transduced cells (transduced with GFP and DNGFR constructs without the TCR). In all cases we were able to subsequently expand the CD8+ T cells with a feeder cell mixture as described by us before (Spits et al., 1982) indicating their functional maturity.

Interestingly irrespective of the HLA-type of CD34+ precursor cells CD8+ single positive T cells expressing only the transduced TCRα or TCRβ chain could be observed (FIGS. 4a and b). The proportion of cells expressing the introduced TCRβ chain was much higher, than that expressing the introduced TCRα chain particularly in the 24 day cultures (FIG. 4a), presumably due to the fact that beta selection favors expansion of cells with an intact TCRβ. Importantly, analysis of the CD8+ T cells that expressed the transduced TCRβ of the HA2-specific TCR and CMV-specific TCR revealed the presence of cells reacting with HLA-A2/HA2 peptide tetramer (FIGS. 4a and b), indicating that the TCRβ chain can pair with endogenous TCRα to form a TCR with the same specificity.

FIGS. 4b and 5a show the differentiation of human CD34+ thymic precursors transduced with a cytomegalo virus (CMV)-specific TCRα and β encoding DNA. The cells that developed in this co-culture were expanded and cloned with a feeder cell mixture as described (Spits et al. 1982) and two expanded CD8+ clones were tested for their cytotoxic activity using a standard 51Cr release assay against HLA-A2+ target cells (The EBV-transformed B cell line ZIJL) that were not treated or transduced with a retroviral construct containing DNA encoding the native antigen (CMV PP65) or loaded with a CMV PP65-derived, HLA-A2-binding, peptide. FIG. 5b shows that two independent cloned cultures derived from co-cultures of TCR-transduced CD34+ thymocytes and OP9DL, were cytotoxic against CMV loaded but not against untreated target cells. As expected, control-transduced cells (transduced with empty GFP and ΔNGFR) were not cytotoxic for CMV PP65-transduced or PP65-derived peptide-loaded target cells (FIG. 5b). These results clearly show that TCR-transduced CD34+ thymocytes can develop into functional cytotoxic T cells following co-culture with OP9DL cells.

### Establishment of T Cell Development in Rag2/Gamma Common Null Mice

We have developed a novel, convenient mouse model that allows for the study of human T cell development and function in an in vivo setting. A robust T cell development was observed upon ip injection of CD34⁺ cells into newborn mice that are deficient for RAG and for the IL-2R gamma chain (now called gamma common). Besides main-stream CD4⁺ and CD8⁺ T cells, all the other subsets of T cells could be observed, including TCRgamma-delta cells, CD3⁺CD56⁺T cells and CD25⁺CD4⁺ cells that could represent T regulatory cells (Gimeno et al., 2004). We also observed development of B cells, NK cells, pDC and monocytes in the circulation and peripheral organs of the mice injected with CD34⁺ cells. In addition, CD15⁺CD11c⁺CD24⁺ granulocytes and a low but consistent percentage of human glycophorin positive cells were present. Together these data show the generation of a rather complete repertoire of human leukocytes in these mice. Similar mice were also made elsewhere and these workers reported that the reconstituted mice could mount an immune response following immunization with tetanus toxoid or with EBV-transformed B cells (Traggiai et al., 2004).

To investigate whether TCR transferred to hematopoietic stem cells would be expressed in the mature T cell progeny, we transduced the HA-2 and the CMV specific receptors into CD34+ cell isolated from fetal liver. New born Rag2/γc null mice were injected with the transduced CD34+ fetal liver cells and 8 weeks later we inspected the thymus of these mice for the presence of T cells expressing the transduced receptors. Importantly, T cells expressing only the TCRβ chain were readily observed in the injected mice (FIG. 6). It is also demonstrated that a considerable proportion of these T cells bind the tetramer for which the original TCR was specific. This observation indicates that the transduced TCRβ pairs with endogeneously formed TCRα to create novel TCR with the same specificity as the TCR from which the TCRβ was derived.

### BRIEF DESCRIPTION OF THE DRAWINGS

FIG. 1 Human T cell development in vitro in co-culture of CD34+ thymic precursor cells and OP9-huDL1 cells

FIG. 2 Transduction of TCRα and β containing retroviral vectors into CD 34+ thymus progenitor cells

FIG. 3 Human CD34+ cells isolated from post-natal thymocytes transduced with defined TCR specificity develop efficiently in the co-culture of CD34+ thymocytes and OP9-huDLl into mature CD8+CD1a-T cells. The development of the TCR transduced T cells is accelerated compared to that of with control transduced CD34+ cells

FIG. 4 Detection of tetramer positive T cells after development of CD34+ thymic precursors transduced with a HA2/HLA-A2 specific and a CMV/HLA-A2-specific TCR

(a) after 24 days of culture with OP9DL1

(b) after 38 days of culture with OP9DL1

FIG. 5 (a) Human CD34+ cells isolated from post-natal thymocytes transduced with TCR specific for CMV PP65 in the context of HLA-A2 develop efficiently in the OP9-huDLl co-culture into mature CD8+CD1a-T cells.

(b) Upon expansion and cloning, the CD8+ T cells generated in the OP9 co-culture, can mediate cytotoxic activity against HLA-A2+ target cells (the EBV-transformed cell line ZIJL) expressing the PP65/HLA-A2 epitope but not against control ZIJL cells that did not express the PP65 epitope (5b).

FIG. 6 Human T cells with chosen TCR specificity develop in vivo in HIS-Rag/yc mice. Shown is the specific tetramer binding in the transduced TCRα+ and TCRβ+ T cells

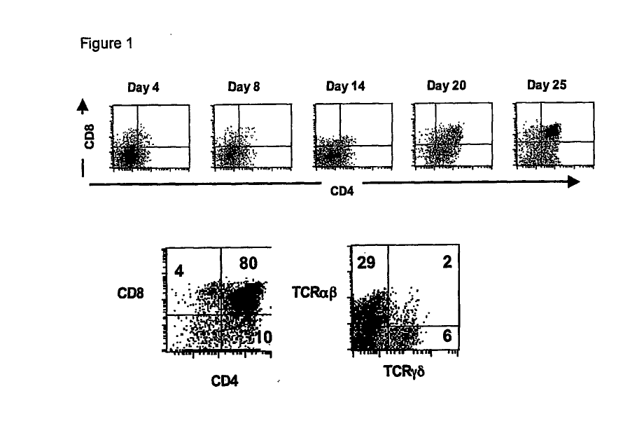

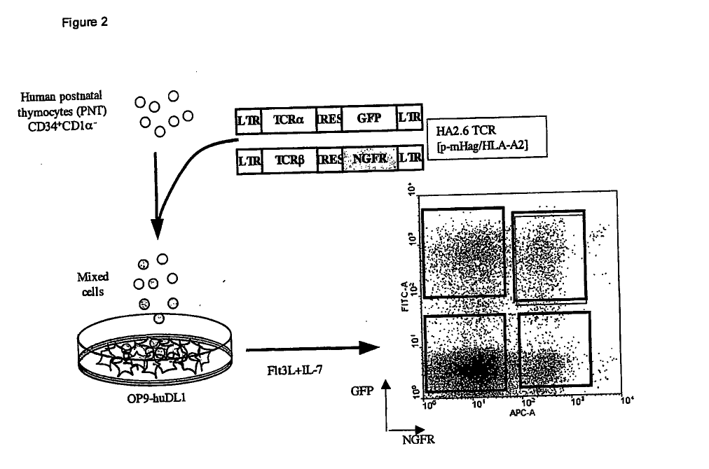

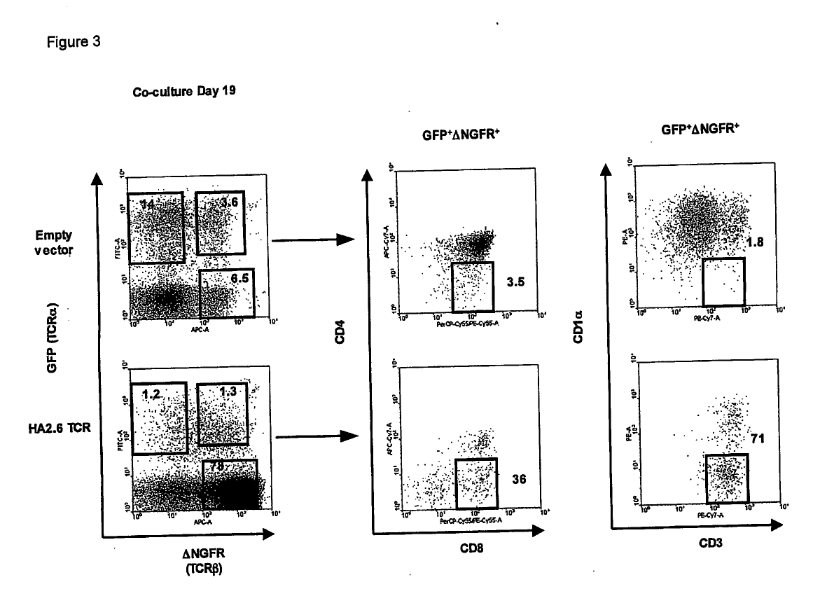

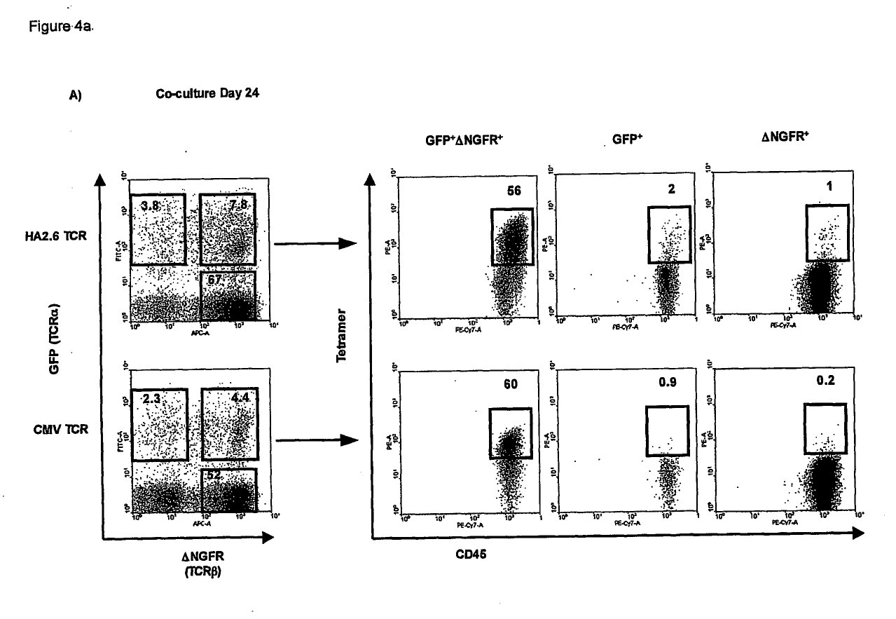

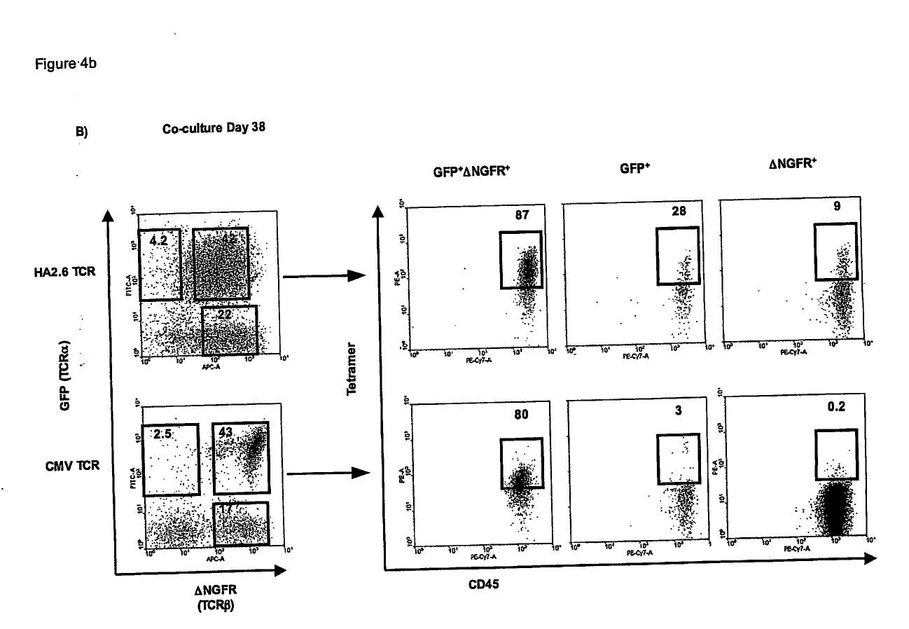

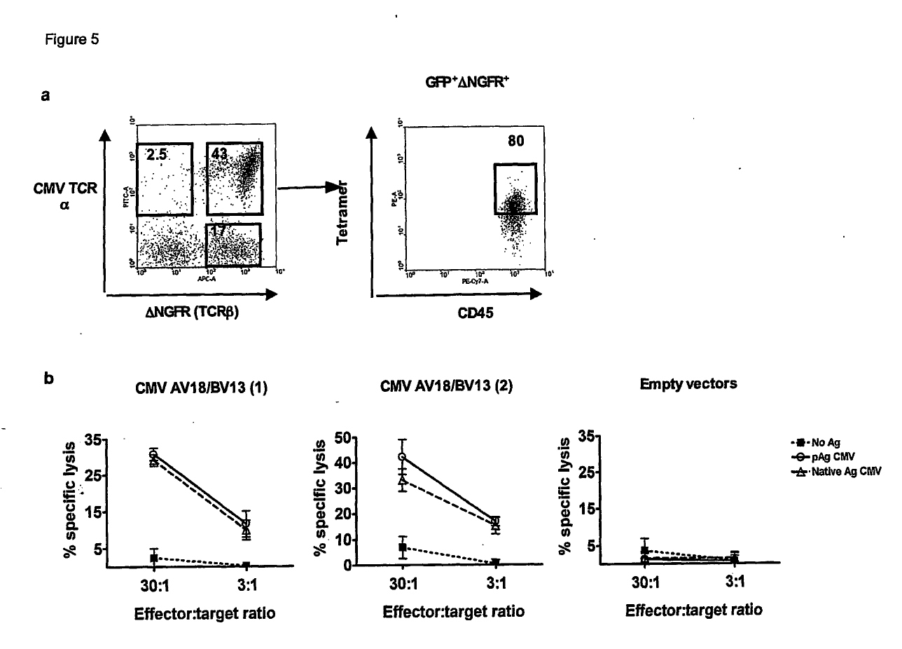

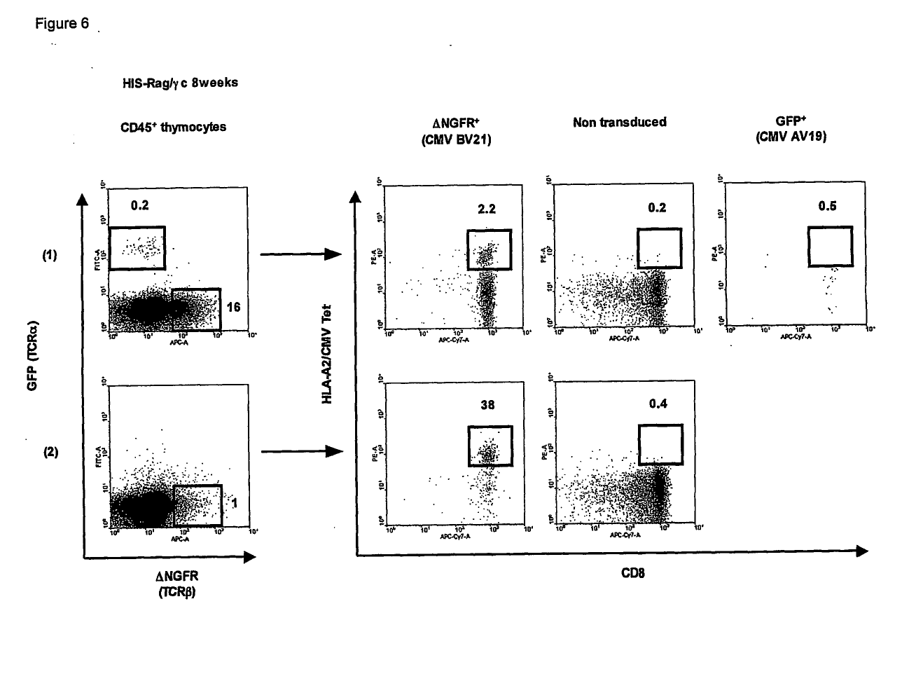

### REFERENCES

Altman, J. D. et al. 1996. Phenotypic analysis of antigen-specific T lymphocytes. Science 274, 94-96.
Gimeno, R., K. Weijer, A. Voordouw, C. H. Uittenbogaart, N. Legrand, N. L. Alves, E. Wijnands, B. Blom, and H. Spits. 2004. Monitoring the effect of gene silencing by RNA interference in human CD34+ cells injected into newborn RAG2−/− gammac−/− mice: functional inactivation of p53 in developing T cells. Blood 104:3886-3893
Holler, P. D., P. O. Holman, E. V. Shusta, S. O'Herrin, K. D. Wittrup, and D. M. Kranz. 2000. In vitro evolution of a T cell receptor with high affinity for peptide/MHC. Proc Natl Acad Sci USA 97:5387-5392
Holler, P. D., L. K. Chlewicki, and D. M. Kranz. 2003. TCRs with high affinity for foreign pMHC show self-reactivity. NatImmunol 4:55-62
Kinsella, T. M., and G. P. Nolan. 1996. Episomal vectors rapidly and stably produce high-titer recombinant retrovirus. Hum Gene Ther 7:1405-1413
Kirberg J, Berns A, von Boehmer H. 1997. Peripheral T cell survival requires continual ligation of the T cell receptor to major histocompatibility complex-encoded molecules. J Exp Med. 186:1269-1275
La Motte-Mohs, R. N., E. Herer, and J. C. Zuniga-Pflucker. 2005. Induction of T-cell development from human cord blood hematopoietic stem cells by Delta-like 1 in vitro. Blood 105:1431-1439
Nakano, T., H. Kodama, and T: Honjo. 1994. Generation of lymphohematopoietic cells from embryonic stem cells in culture. Science 265:1098-1101.
Res, P., E. Martinez-Caceres, A. Cristina Jaleco, F. Staal, E. Noteboom, K. Weijer, and H. Spits. 1996. CD34+CD38dim cells in the human thymus can differentiate into T, natural killer, and dendritic cells but are distinct from pluripotent stem cells. Blood 87:5196-5206
Schneck J P. 2000. Monitoring antigen-specific T cells using MHC-Ig dimers. Immunol Invest. May; 29(2):163-9
Spits, H., H. Ijssel, C. Terhorst, and J. E. de Vries. 1982. Establishment of human T lymphocyte clones highly cytotoxic for an EBV-transformed B cell line in serum-free medium: isolation of clones that differ in phenotype and specificity. J Immunol 128:95-99
Traggiai, E., L. Chicha, L. Mazzucchelli, L. Bronz, J. C. Piffaretti, A. Lanzavecchia, and M. G. Manz. 2004. Development of a human adaptive immune system in cord blood cell-transplanted mice. Science 304:104-107
Weijer K, Uittenbogaart C H, Voordouw A, Couwenberg F, Seppen J, Blom B, Vyth-Dreese F A, Spits H.2002. Intrathymic and extrathymic development of human plasmacytoid dendritic cell precursors in vivo. Blood 99:2752-2759
Willemsen, R. A., R. Debets, E. Hart, H. R. Hoogenboom, R. L. Bolhuis, and P. Chames. 2001. A phage display selected fab fragment with MHC class I-restricted specificity for MAGE-A1 allows for retargeting of primary human T lymphocytes. Gene Ther 8:1601-1608
Yssel, H., De Vries, J. E. Koken, M. Van Blitterswijk, W, Spits, H. 1994. Serum-free medium for generation and propagation of functional human cytotoxic and helper T cell clones. J. Immol Methods 72: 219-227

1. A method for generating a T cell comprising a T cell receptor (TCR) capable of specifically binding an antigen of interest, the method comprising:
providing a hematopoietic stem cell and/or a precursor cell of a T cell with a nucleic acid sequence comprising at least part of a rearranged gene encoding a TCR chain, or a functional equivalent thereof; and
allowing for differentiation of said hematopoietic stem cell and/or precursor cell and generation of at least one T cell derived from said hematopoietic stem cell and/or precursor cell.

2. The method according to claim 1, further comprising at least in part isolating at least one T cell derived from said hematopoietic stem cell and/or precursor cell.

3. The method according to claim 1, wherein said at least part of a rearranged gene is derived from a first T cell receptor, said first T cell receptor being capable of binding said antigen of interest.

4. The method according to claim 1, comprising selecting a T cell comprising a T cell receptor with a desired property.

5. The method according to claim 3, comprising selecting a T cell comprising a T cell receptor which is capable of binding said antigen of interest with a higher affinity as compared to said first T cell receptor from which said at least part of a rearranged gene is derived.

6. The method according to any claim 1, wherein said hematopoietic stem cell and/or precursor cell comprises a human stem cell and/or precursor cell.

7. The method according to claim 1, wherein said hematopoietic stem cell and/or precursor cell and said rearranged gene encoding at least a functional part of a TCR chain are derived from the same species.

8. The method according to claim 1, wherein said hematopoietic stem cell and/or precursor cell and said rearranged gene are human.

9. The method according to claim 1, wherein said precursor cell comprises a CD34+ precursor cell.

10. The method according to claim 1, wherein said hematopoietic stem cell and/or precursor cell is provided with a nucleic acid sequence comprising a rearranged beta chain of a T cell receptor.

11. The method according to claim 1, wherein said hematopoietic stem cell and/or precursor cell is provided with a nucleic acid sequence comprising a rearranged alpha chain of a T cell receptor.

12. The method according to claim 1, wherein a plurality of T cells derived from said hematopoietic stem cell and/or precursor cell is obtained.

13. A method according to claim 12, wherein a plurality of T cells with different antigen binding affinities is obtained.

14. The method according to claim 1, wherein said T cell receptor is generated in vitro.

15. The method according to claim 1, wherein said T cell receptor is generated in vivo.

16. The method according to claim 15, wherein a non-human animal is provided with a human hematopoietic stem cell and/or a human precursor cell of a T cell, said hematopoietic stem cell and/or precursor cell being provided with said nucleic acid sequence comprising said at least part of a rearranged gene encoding a TCR chain or a functional equivalent thereof.

17. A method according to claim 16, wherein said non-human animal is provided with said antigen of interest after said at least one T cell is generated.

18. The method according to claim 1, wherein said antigen of interest comprises a human antigen.

19. The method according to claim 1, wherein said antigen of interest comprises at least an immunogenic part of a human autoantigen, a tumor-associated antigen and/or an antigen expressed on malignant cells.

20. The method according to claim 1, wherein a T cell capable of specifically binding an antigen of interest is obtained with a binding assay using said antigen of interest or a functional part, derivative and/or analogue thereof.

21. The method according to claim 16, wherein said non-human animal is essentially devoid of at least one kind of endogenous hematopoietic cell.

22. The method according to claim 16, wherein said non-human animal is essentially devoid of endogenous B cells, endogenous T cells, and/or endogenous natural killer (NK) cells.

23. The method according to claim 16, wherein said non-human animal comprises a mouse.

24. A method according to claim 23, wherein said mouse comprises a RAG2^{-/-}γc^{-/-} mouse.

25. The method according to claim 16, wherein said hematopoietic stem cell and/or precursor cell is administered to said non-human animal within 1 week after birth.

26. The method according to claim 1, wherein said hematopoietic stem cell and/or precursor cell is transduced with a lentiviral vector comprising said nucleic acid sequence comprising said rearranged gene.

27. A T cell obtainable by the method according to claim 1.

28. A library comprising a plurality of T cells comprising T cell receptors with different antigen binding affinities obtainable by the method according to claim 1.

29. A method for selecting a T cell comprising a T cell receptor capable of specifically binding an antigen of interest, comprising contacting said antigen of interest or a functional part, derivative and/or analogue thereof with a library according to claim 28.

30. The method according to claim 1, further comprising:
obtaining a nucleic acid sequence encoding said T cell receptor; and
providing a suitable host cell with said nucleic acid sequence.

31. A method according to claim 30, wherein said host cell is a human T cell.

32. An isolated host cell capable of specifically binding an antigen of interest obtainable by the method according to claim 30.

33. An isolated stem cell and/or precursor cell of a T cell, said stem cell and/or precursor cell being provided with a nucleic acid sequence comprising at least part of a rearranged gene encoding a TCR chain of a T cell receptor, or a functional equivalent thereof.

34. A cell according to claim 33, wherein said T cell receptor is capable of specifically binding at least an immunogenic part of a human autoantigen, a tumor-associated antigen and/or an antigen expressed on malignant cells.

35. A non-human animal comprising the cell of claim 33.

36. A non-human animal according to claim 35, which is a RAG2^{-/-}γc^{-/-} mouse.

37. (canceled)

38. A method of treating a tumor-related disease in a subject, the method comprising:
utilizing a host cell according to claim 32 in the treatment of a tumor-related disease in the subject.

39. A method for providing a T cell with the capability of binding an antigen of interest with a desired affinity, the method comprising:
providing said T cell with a nucleic acid sequence encoding at least part of a T cell receptor, or a functional equivalent thereof, obtainable by the method according to claim 1.

40. A T cell capable of binding an antigen of interest with a desired affinity obtainable by a method according to claim 39.

41. A method for providing an individual with an capability or enhanced capability of generating an immune response against an antigen of interest, the method comprising:
providing said individual with at least one T cell and/or host cell according to claim 27.

42. A method according to claim 41, wherein said T cell and/or host cell is derived from said individual.

43. The method according to claim 41, wherein said individual is matched for an HLA molecule that is utilized by said T cell and/or host cell.

44. A method of generating a modified T cell comprising a T cell receptor able to specifically bind an antigen of interest, the method comprising:
providing a cell selected from the group consisting of a hematopoietic stem cell, a precursor cell of a T cell, and a combination thereof, with a nucleic acid sequence comprising at least part of a rearranged gene encoding a T cell receptor (TCR) chain from a first T cell receptor able to bind the antigen of interest; and
differentiating the cell provided with the nucleic acid sequence
so as to generate the modified T cell comprising a T cell receptor able to specifically bind the antigen of interest.

45. A cell selected from the group consisting of a stem cell, a precursor cell of a T cell, and combinations thereof, wherein said cell comprises:
a recombinant nucleic acid sequence comprising at least part of a rearranged gene encoding a T cell receptor (TCR) chain of a T cell receptor.
